(19) United States
(12) Patent Application Publication
(10) Pub. No.: US 2010/0069971 A1
(43) Pub. Date: Mar. 18, 2010

(54) PEDICLE SCREW SURFACE TREATMENT FOR IMPROVING BONE-IMPLANT INTERFACE

(76) Inventor: Uri Arnin, Kiryat Tivon (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 12/513,668

(22) PCT Filed: Nov. 14, 2007
(86) PCT No.: PCT/IL2007/001413

§ 371 Date: May 6, 2009

Related U.S. Application Data
(60) Provisional application No. 60/865,876, filed on Nov. 15, 2006.

(51) Int. Cl. A61B 17/86; B24C 1/00

(52) U.S. Cl. 606/301; 451/36

(57) ABSTRACT

A method for surface treatment of a pedicle screw including roughening a surface of a pedicle screw by blasting the surface with a Resorbable Blast Media (RBM).

### FIELD OF THE INVENTION

The present invention is related generally to surface treatment of pedicle screws to improve a bone-implant interface.

### BACKGROUND OF THE INVENTION

One main concern when using pedicle screw based implants is screw loosening. The loads acting on the human spine during normal daily life transfer a variety of forces and moments to the pedicle screws that may lead to micro-motions in the bone-screw interface and delay or prevent the fixation of the screw to the bone.

In the general orthopedic field, one of the most common ways to improve osseointegration is by the addition of HA (Hydroxyapatite) coating, which is in clinical use since 1986. The short term results of the coating showed high bone to implant contact percentage, but over the mid and long terms two main disadvantages of the HA coating were presented. It was found that the first generation of HA tends to resorb and delaminate, leading to poor attachment between the implant and the bone. Today, a second generation of HA coating is used, but there is still a great lack of certainty in terms of long-term results. A third generation of HA nano-coating is now in research and development.

### SUMMARY OF THE INVENTION

The present invention seeks to provide a surface treatment of pedicle screws so as to improve the interface between bone and implant, as is described more in detail hereinbelow.

There is thus provided in accordance with an embodiment of the present invention a method for surface treatment of a pedicle screw including roughening a surface of a pedicle screw by blasting the surface with a Resorbable Blast Media (RBM). In one example, the pedicle screw is constructed of a titanium alloy and the surface is blasted with calcium phosphate particles.

In accordance with an embodiment of the present invention, the method includes passivating the surfaced after RBM blasting without acid etching.

The surface of the pedicle screw includes crests and valleys of screw threads, a shank of the pedicle screw, a (polyaxial) head of the pedicle screw, or any combination thereof.

The surface of the pedicle screw can be blasted to approximately a 2, 3 or 3.34 μm average roughness.

### DETAILED DESCRIPTION OF EMBODIMENTS

In accordance with an embodiment of the present invention, surfaces of pedicle screws are roughened by using Resorbable Blast Media (RBM), such as by blasting with calcium phosphate. This method creates a textured surface by blasting a machined titanium alloy implant with calcium phosphate particles, which is then passivated, without acid etching, to remove residual media. The surfaces of the pedicle screws which are roughened include the crests and valleys of the screw threads, the screw shank and the screw head (including polyaxial screw heads). Since there is no acid etching, the titanium screw material is not susceptible to titanium grain boundary degradation that can occur during aggressive acid etching.

The following are successful test results of in-vitro and in-vivo testing.

In-Vitro Study:

Several disks (15 mm in diameter and 1 mm thick) made of Ti-6Al-4V were prepared to fit into 24-well plates. Surfaces were either machined to produce 0.2 μm smooth surfaces, or grit blasted to result in 2, 3 or 3.34 gm roughness. Roughness refers to average roughness (RA), which is the average height of the bumps on a surface, measured in micrometers (μm). Surface morphology and chemistry were characterized by scanning electron microscopy (SEM), X-ray photon spectroscopy (XPS) and energy dispersive analysis of X-rays (EDAX). Human osteoblast-like cells were cultured on the disks as well as on tissue cultured treated polystyrene (plastic) until cells reach confluence on plastic. The effect of Ti-6Al-4V surface microstructure on TGF-β1 level of MG63 osteoblast-like cells was also studied. Total and active TGF-β1 levels were measured separately. Data were analyzed by ANOVA (Analysis of Variance) and significant differences between groups determined using the Bonferroni modification of Student's t-test. (p<0.05, v. plastic; p<0.05, v. smooth Ti-6Al-4V; p<0.05, v. Ti-6Al -4V surface with Ra of 3 μm)

Responses of cells to different roughed metal alloy surfaces were analyzed and the results are summarized in the following table:

| | ng Osteocalcin/ | ng Osteocalcin/ | ng |
|---|---|---|---|
| Material | Well | Cell | TGF-β1/Cell |
| Plastic | <6 | ~3 × 10⁻⁵ | ~3 × 10⁻⁵ |
| Ti—6Al—4V 0.2 μm | <6 | ~5 × 10⁻⁵ | ~3 × 10⁻⁵ |
| Ti—6Al—4V 2 μm | ~7 | ~25 × 10⁻⁵ | ~12 × 10⁻⁵ |
| Ti—6Al—4V 3 μm | ~8 | ~30 × 10⁻⁵ | ~14 × 10⁻⁵ |
| Ti—6Al—4V 3.34 μm | >9 | ~60 × 10⁻⁵ | ~30 × 10⁻⁵ |

It is clear the osteocalcin and Transforming Growth Factor (TGF) level increased in a surface dependent manner, with highest values seen on roughest surfaces.

The results show that roughening of surfaces by RBM promotes the differentiated of osteoblast, inhibit osteoclast cells activity and actively participate in bone remodeling.

In-Vivo Study:

Pedicle screws (ø5×25 mm) made of Ti-6Al-4V were implanted into L4 and L5 vertebra of 15 sheep. Fusion rods were connected vertically to attain proper fixation and load bearing. Each sheep received either 4 smooth (Ra=0.2 μm) or 4 rough (Ra=3 μm) screws.

The animals were euthanized after 12 weeks of implantation, the screws in L4 vertebras were sent to histomorphometric analysis to evaluate the Bone-Implant-Contact (BIC) percentage and the screws in L5 vertebras were measured for the required torque to remove them.

The results are summarized in the following table, which shows BIC and removal torque results after 3 months of implantation:

| | Smooth | Rough |
|---|---|---|
| | Ra = 0.2 μm | Ra = 3 μm |
| BIC % | 60.29 ± 4.78 | 80.99 ± 4.42 |
| EBIC % | 83.67 ± 1.74 | 81.35 ± 2.96 |
| BV % | 78.07 ± 1.47 | 79.45 ± 1.60 |
| Removal Torque | 2.28 ± 0.32 | 5.29 ± 0.41 |
| (N-m) | | |

The results of the histomorphological analysis in the above table clearly show that average BIC % around rough surface Ti-6Al-4V implant was significantly higher than that around smooth ones. There were no difference of expected bone to implant contact (EBIC %) and bone volume (BV %) of two groups. The required force to remove screws from the bone showed also significant higher values for rough implants than smooth implants. In short, the roughened surfaces resulted in more than twice removal torque value and a growth of 34% in the bone-implant-contact.

1. A method for surface treatment of a pedicle screw comprising:
roughening a surface of a pedicle screw by blasting the surface with a Resorbable Blast Media (RBM), and comprising passivating said surface after RBM blasting without acid etching.

2. The method according to claim 1, comprising blasting said surface with calcium phosphate particles.

3. The method according to claim 1, wherein said pedicle screw is constructed of a titanium alloy and said surface is blasted with calcium phosphate particles.

4-5. (canceled)

6. The method according to claim 1, wherein said surface of said pedicle screw comprises at least one of crests and valleys of screw threads.

7. The method according to claim 1, wherein said surface of said pedicle screw comprises at least one of a shank and a head of said pedicle screw.

8. The method according to claim 1, wherein said surface of said pedicle screw comprises a polyaxial head of said pedicle screw.

9. The method according to claim 1, wherein said surface of said pedicle screw is blasted to approximately a 2 μM average roughness.

10. The method according to claim 1, wherein said surface of said pedicle screw is blasted to approximately a 3 μm average roughness.

11. The method according to claim 1, wherein said surface of said pedicle screw is blasted to approximately a 3.34 μm average roughness.

12. An article comprising:
a pedicle screw comprising a surface roughened by blasting the surface with a Resorbable Blast Media (RBM), and passivating said surface after RBM blasting without acid etching.

13. The article according to claim 12, wherein said surface of said pedicle screw has approximately a 3.34 μm average roughness.
